(19) United States
(12) Patent Application Publication
(10) Pub. No.: US 2022/0008743 A1
(43) Pub. Date: Jan. 13, 2022

(54) DUAL-HORN SUBLINGUAL LIGHT THERAPY DEVICE

(71) Applicant: PhotonX TherapeutX, Inc., Oldsmar, FL (US)

(72) Inventor: Scot Johnson, Tampa, FL (US)

(21) Appl. No.: 17/182,627

(22) Filed: Feb. 23, 2021

Related U.S. Application Data
(60) Provisional application No. 62/980,986, filed on Feb. 24, 2020.

(51) Int. Cl. A61N 5/06

(52) U.S. Cl. CPC A61N 5/0603; A61N 2005/063; A61N 2005/0606

(57) ABSTRACT

A dual-horn sublingual light therapy device useful for a variety of applications. In one embodiment of the inventive concept, a sublingual light therapy device includes a light conduit connected to a mouthpiece, with one end of the light conduit connected to a light source and the mouthpiece disposed directly under the tongue of a patient. The mouthpiece may comprise a rigid sublingual photonic applicator disposed inside a compliant sublingual light therapy sleeve.

### FIELD OF THE DISCLOSURE

The present disclosure generally relates to devices and methods for providing therapy using light.

### BRIEF SUMMARY OF THE DISCLOSURE

The subject matter presented herein provides a sublingual light therapy device useful for a variety of applications. In one embodiment of the inventive concept, a sublingual light therapy device includes a light conduit connected to a mouthpiece, with one end of the light conduit connected to a light source and the other end disposed directly under the tongue of a patient.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

The foregoing disclosure will be best understood, and the advantages thereof made most clearly apparent, when consideration is given to the following detailed description in combination with the drawing figures presented. The detailed description makes reference to the following drawings:

FIG. 1A shows a sublingual light therapy assembly from a top view;

FIG. 1B shows the sublingual light therapy assembly of FIG. 1A from a side view;

FIG. 1C shows the sublingual light therapy assembly of FIGS. 1A and 1B from a bottom view;

FIG. 1D shows the sublingual light therapy assembly of FIGS. 1A-1C from a first oblique view;

FIG. 1E shows the sublingual light therapy assembly of FIGS. 1A-1D from a second oblique view;

FIG. 1F shows the sublingual light therapy assembly of FIGS. 1A-1F from a third oblique view;

FIG. 2A shows a sublingual light therapy applicator from a first point of view;

FIG. 2B shows the sublingual light therapy applicator of FIG. 2A from a second point of view;

FIG. 3A shows the sublingual light therapy applicator of FIGS. 2A and 2B as viewed from the top;

FIG. 3B shows the sublingual light therapy applicator of FIGS. 2A, 2B and 3A as viewed from the side;

FIG. 3C shows the sublingual light therapy applicator of FIGS. 2A, 2B, 3A and 3B as viewed from the bottom;

FIG. 3D shows the sublingual light therapy applicator of FIGS. 2A, 2B and 3A-3C as viewed from one end;

FIG. 3E shows the sublingual light therapy applicator of FIGS. 2A, 2B and 3A-3D as viewed from a second end opposite the end shown in FIG. 3D;

FIG. 3F shows the sublingual light therapy applicator of FIGS. 2A, 2B and 3A-3E as viewed from an oblique angle;

FIG. 4A shows a sublingual light therapy assembly, comprising the sublingual light therapy applicator of FIGS. 2A, 2B and 3A-3E in combination with a fiber optic assembly, as viewed from a first end thereof;

FIG. 4B shows the sublingual light therapy assembly of FIG. 4A, as viewed from the top;

FIG. 4C shows the sublingual light therapy assembly of FIG. 4A, as viewed from an oblique angle;

FIG. 5A shows a sublingual light therapy sleeve from a first point of view;

FIG. 5B shows the sublingual light therapy sleeve of FIG. 5A from a second point of view;

FIG. 5C shows the sublingual light therapy sleeve of FIGS. 5A and 5B viewed from the top thereof;

FIG. 5D shows the sublingual light therapy sleeve of FIGS. 5A-5C viewed from the side thereof;

FIG. 5E shows the sublingual light therapy sleeve of FIGS. 5A-5D viewed from the bottom thereof;

FIG. 5F shows the sublingual light therapy sleeve of FIGS. 5A-5E viewed from a first end thereof;

FIG. 5G shows the sublingual light therapy sleeve of FIGS. 5A-5F viewed from a second end opposite the end shown in FIG. 6D;

FIG. 5H shows the sublingual light therapy sleeve of FIGS. 5A-5G viewed from an oblique angle;

FIG. 5I shows the sublingual light therapy sleeve of FIGS. 5A-5H viewed from an oblique angle;

FIG. 5J shows the sublingual light therapy sleeve of FIGS. 5A-5I viewed from an oblique angle;

FIG. 5K shows the sublingual light therapy sleeve of FIGS. 5A-5J viewed from an oblique angle; and

FIG. 5L shows the sublingual light therapy sleeve of FIGS. 5A-5K viewed from an oblique angle.

### DETAILED DESCRIPTION

A device for light therapy is provided herein. The following detailed description provides certain specific embodiments of the subject matter disclosed herein. Although each embodiment represents a single combination of elements, the subject matter disclosed herein should be understood to include sub-combinations of the disclosed elements. Thus, if one embodiment comprises elements A, B, and C, and a second embodiment comprises elements B and D, then the inventive subject matter is also intended to include other remaining combinations of A, B, C, or D, even if not explicitly disclosed herein.

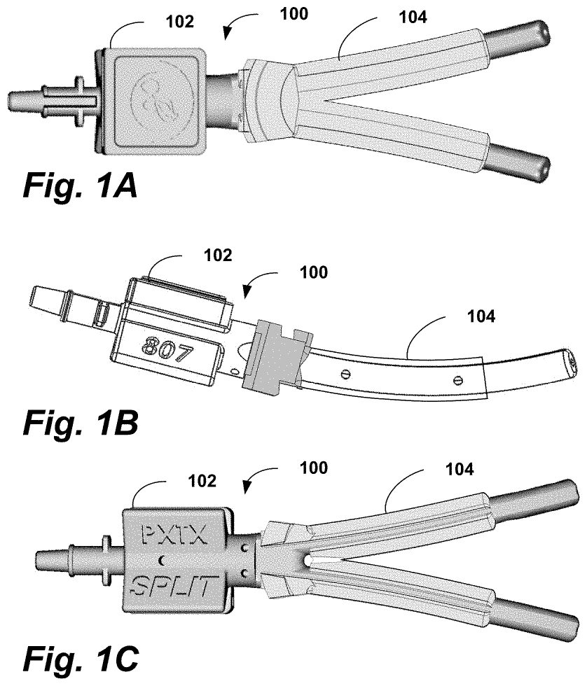

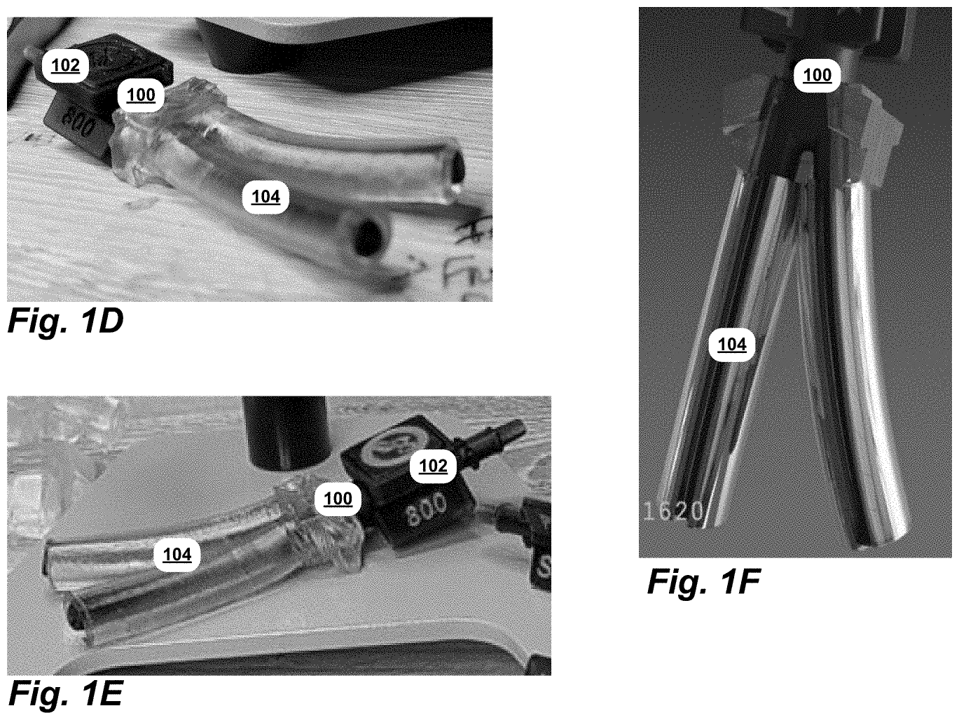

FIGS. 1A-1F show sublingual light therapy assembly 100 as viewed from various points of view. As seen in these figures, sublingual light therapy assembly 100 comprises a rigid sublingual photonic applicator 102 disposed inside a compliant sublingual light therapy sleeve 104. The structure and function of sublingual photonic applicator 102 and sublingual light therapy sleeve 104 are discussed in further detail below.

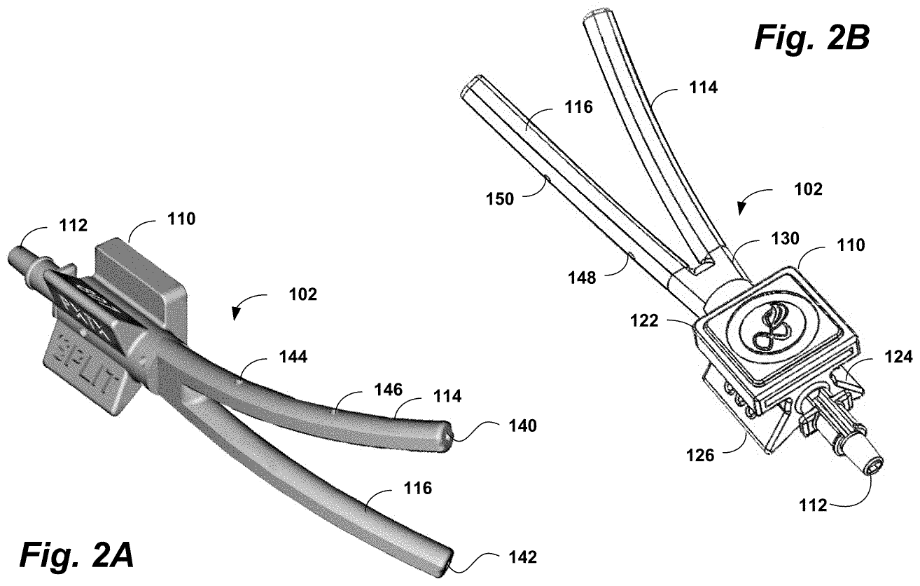

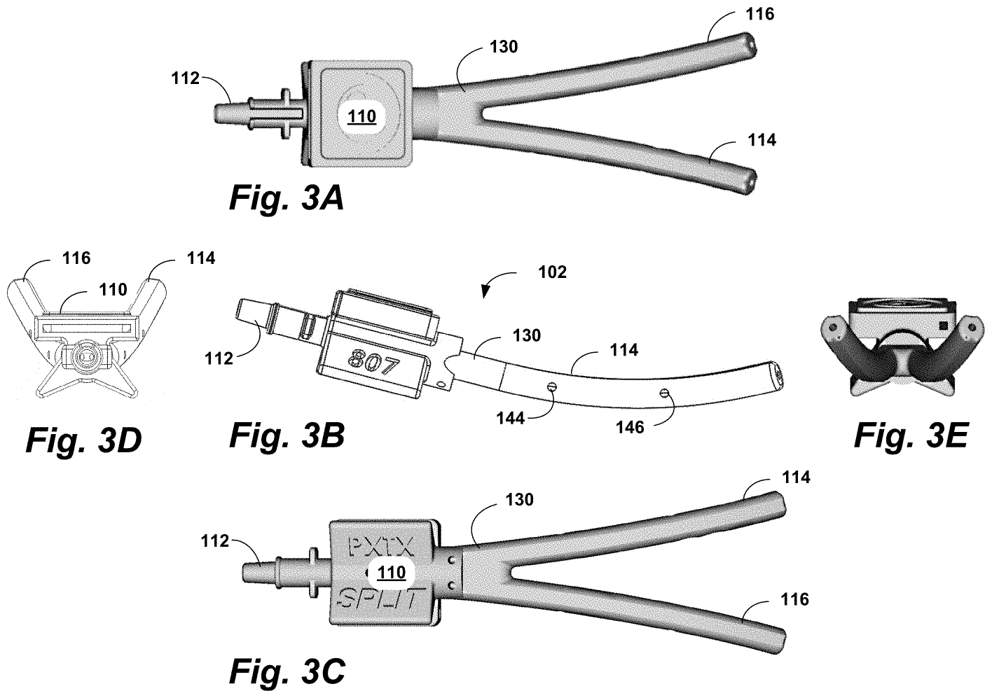

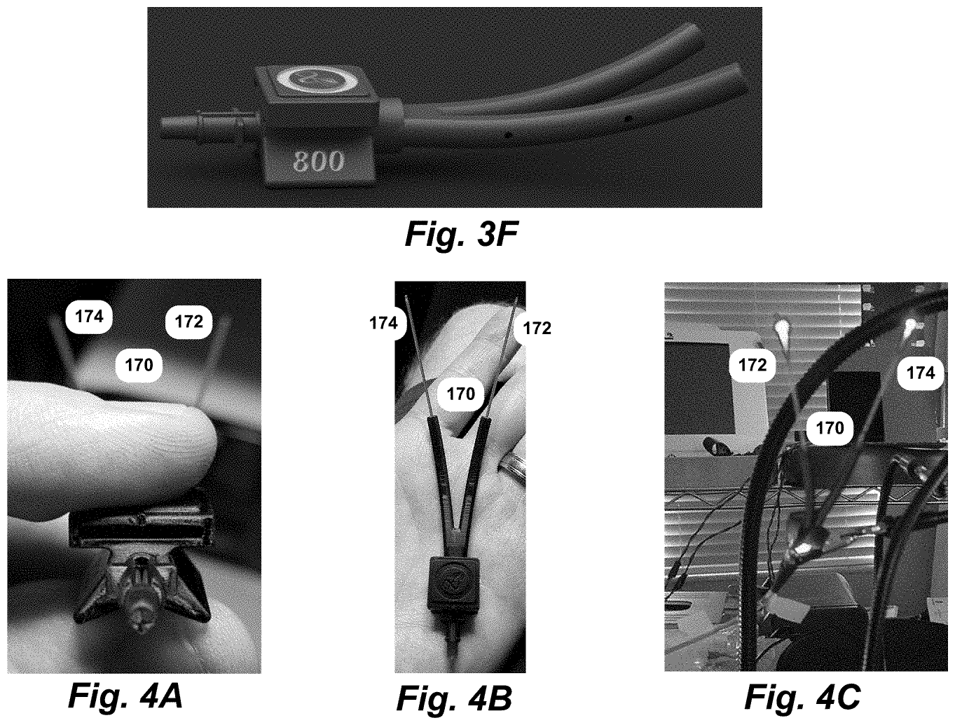

FIGS. 2A, 2B and 3A-3F show the sublingual light therapy applicator 102 viewed from various points of view. As seen in these figures, sublingual light therapy applicator 102 comprises a main body 110 having an optical connector 112 on one side thereof and a pair of optical horns 114, 116 opposite optical connector 112. Main body 110 incorporates a generally-cylindrical central core 120 having a generally-rectangular upper landing 122 on the top thereof and a pair of prismatic wings 124, 126 extending downwardly from each side thereof. The shape and size of optical connector 112 will be dictated by the characteristics of the optical components to which optical connector 112 is designed to interface.

Optical horns 114, 116 are connected to main body 110 via optical manifold 130, which has a branched “V” shape, with optical horn 114 attached to one leg of optical manifold 130 and optical horn 116 attached to the other leg of optical manifold 130. Each optical horn 114, 116 has a curved shape and a generally-circular cross-section.

The embodiment shown in these figures is specifically designed to be manufactured by 3D printing, but may be manufactured by injection molding or another suitable manufacturing method. The material may vary, as well. In certain embodiments, the material may be a biocompatible resin sufficient for use for relatively short periods of time. Such short periods of time may vary from less than 10 minutes or up to 4 hours.

Central apertures 140, 142 run axially down the center of optical horns 114 and 116, respectively. An array of transverse apertures 144, 146, 148, 150 are disposed in the sides of optical horns 114, 116. Transverse apertures 144, 146, 148, 150 serve as drain holes for 3D printing. Alternate embodiments may incorporate more or fewer transverse apertures, along with other apertures, such as those shown in FIG. 1C. These apertures are placed strategically to keep the vacuum pressure as low as possible during manufacturing, in order to facilitate proper formation of the optical horns 114, 116. Optical fibers fit through central apertures 140, 142 with only the ends polished, otherwise keeping their core, cladding and buffer. In the event that a buffer is pinched or bunched up, the higher light carrying modes of a multi-mode fiber may be lost.

FIGS. 4A-4C show a sublingual light therapy assembly 170, comprising the sublingual light therapy applicator 102 of FIGS. 2A, 2B and 3A-3E in combination with a fiber optic assembly 170, as viewed from a variety of angles. Fiber optic assembly 170 comprises a pair of fiber optics 172, 174 extending from optical connector 112 through optical horns 114, 116, respectively.

Using this angled geometry, the horns 114, 116 of sublingual light therapy assembly 170 are aligned so that one of them comes up under the tongue on the lingual artery side, shining light up and into the lingual artery through the thinnest part of the membrane separating the inner mouth and the tissue/vasculature within the tongue. This region of the membrane is most transmissive for the purposes of photon infusion therapy. The lingual artery itself carries nutrient-rich, freshly-oxygenated blood from the carotid artery, which blood is the primary photo-receptor intended for sublingual photonic infusion therapy. The other horn comes up similarly under the opposite side of the tongue, shining right up into the lingual vein. This provides the most strategic site to create photo-excitation of the desired chromophores, including cytochrome C oxidase. The design of sublingual light therapy assembly 100 scoops at first under the tongue, and then curves up to meet the tongue at its lower surfaces, including the sublingual mucosal membrane, the tongue root, and the tongue itself.

A variety of embodiments may be envisioned from the disclosure herein. In one illustrative embodiment, the two horns 114, 116 each contain one 800 um core fiber optic 172, 174. The two fiber optics 172, 174 come together at the optical connector 112. The two fiber end faces at the input side of optical connector 112 are adjacent to one another and connected to an input fiber optic cable carrying a single fiber optic. Through the tapered adapter, an SMA905 fiber optic cable carrying photonic energy may be brought to within the same or similar parameters as is provided by a standard SMA905 adapter, thus providing stable and low dB photonic attenuation.

In certain embodiments, the conical emission from the single fiber from the photon-carrying input fiber optic cable on the two adjacent fiber faces at the input side of sublingual light therapy assembly 100. The two fiber faces of 172, 174 then carry that signal to the tips of the two horns 114, 116. In an alternate embodiment, the fiber optic cable carrying the original photonic energy is itself a side-by-side two-fiber patch cable with a standard connector terminator, such as a standard SMA905 connector. Two adjacent fibers at the input side of sublingual light therapy assembly 100 are broken out with fusion splicing to two individual SMA905 fiber patch cables. Alternately, the two fibers at the input side of sublingual light therapy assembly 100 do not get broken out. The fibers may stay side by side throughout the patch cable.

Applicant has conducted experiments using a standard commercially-available bifurcated patch cable. Through experimentation, Applicant has learned that the entire face of an SMA905 connector may be saturated uniformly with photonic energy by focusing the energy on a focal point within the fiber slightly behind the face. Accordingly, whether a single 600 um fiber or dual 600 um fibers are used in the patch cable going to the end-effector/applicator, their faces parallel to each other and the fibers adjacent to each other, each fiber in either scenario is carrying equal power. A single 600 um fiber going from the photon source to an applicator carries X uW/cm2 irradiance. Within sublingual light therapy assembly 100, that photonic energy is transferred theoretically in equal parts to each fiber, plus losses, split between the two end faces of the two horns 114, 116. A double fiber patch cable carries 2X the power as the single core patch cable. The double core, when aligned with sublingual light therapy assembly 100 properly, transfers 2X power minus losses, so that each horn ends up delivering nearly X amount of power.

From the input side of the applicator, the two fibers 170, 172, which may be 800 um core, high or low OH depending on the application, begin to bend outwards parallel to the bottom of the applicator in a smooth circular arc at no less than the LONG TERM minimum bend radius. This is both to ensure the fiber will not sustain damage through prolonged over bending, and to ensure that the approach regarding overfilled launch is as effective as possible. This design relies in large part on cladding modes for maximum light throughput.

Owing to the fact that the present device utilizes relatively short lengths of fiber, light losses to microbending and length-related optical attenuations are not a significant concern so long as proper design recommendations are followed. The device does experience microbending losses if minimum bend radius recommendations are not followed. For an 800 um fiber used in the present disclosure, the minimum bend radius is specified as 80 mm. In one exemplary embodiment of the present disclosure, the outward radius of horns 114, 116 is approximately 100 mm. At the same time, the horns are radiused upwards at a radius of approximately 80 mm. The forked geometry contours quite comfortably along the frenelum under the tongue, which ensures comfortable use, consistent positioning, and patient compliance. The device may be reusable, semi-reusable or disposable, depending on the application.

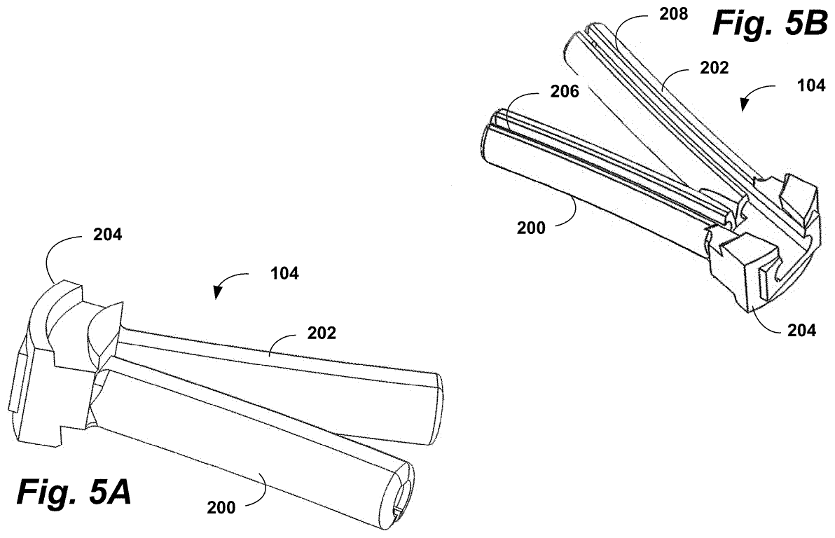

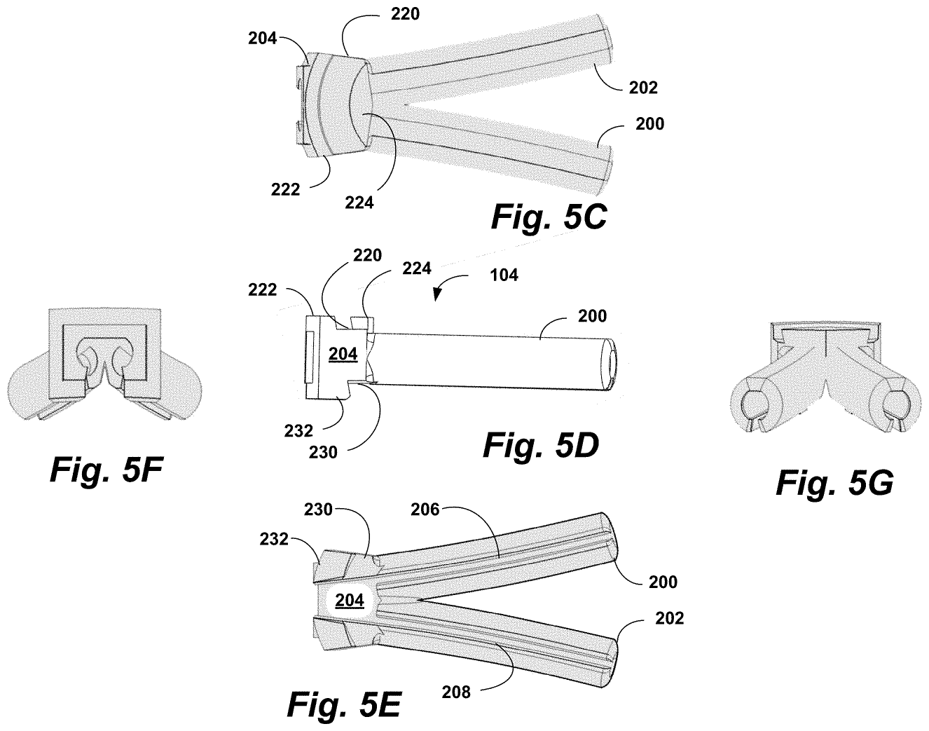

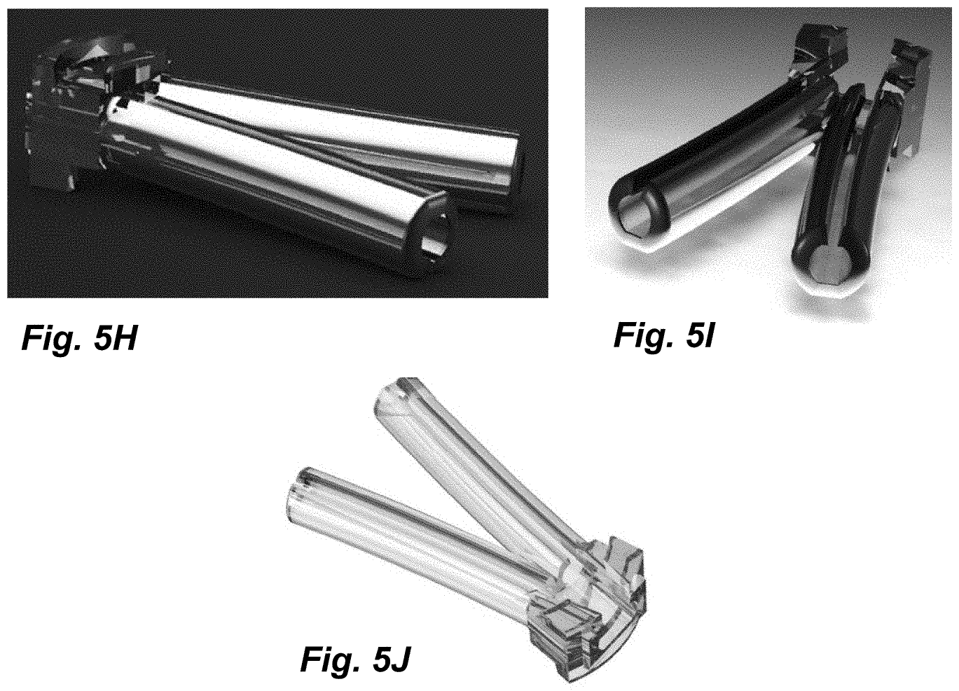

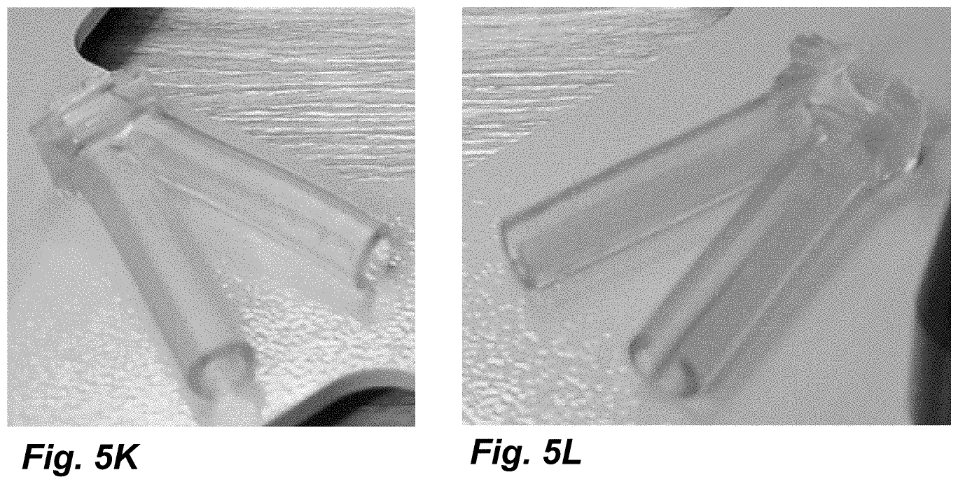

FIGS. 5A-5L show the geometry of sublingual light therapy sleeve 104 from various points of view. As seen in these figures sublingual light therapy sleeve 104 comprises a pair of sleeve segments 200, 202 connected to one together at central joint 204. Each of sleeve segments 200, 202 has a generally-c-shaped cross-section along its length. A pair of channels 206, 208 run along the bottom edge of sleeve segments 200, 202 to facilitate the insertion of the relatively rigid sublingual light therapy applicator 102 into the relatively compliant sublingual light therapy sleeve 104.

Central joint 204 has a complex shape defined generally as a segment of an arcuate profile. On the upper portion of central joint 204, upper dental channel 220, having a generally rectangular cross-section, follows a generally-radial path about a vertical axis, separating the uppermost surface of central joint into a front upper radial ridge 222 and an upper rear land 224. On the bottom of central joint 204, lower rear dental land 230 sits behind front lower radial ridge 232. Lower rear dental land 230 has a generally-radial shape similar to, but set back from, upper dental channel 220. As the name suggests, upper dental channel 220 is shaped and sized to interface with the upper front teeth of a patient, while lower rear dental land 230 is shaped and sized to interface with the lower front teeth of the patient. In the embodiment shown in FIGS. 5A-5L, lower rear dental land 230 is set back from upper dental channel 220 in order to angle sublingual light therapy sleeve 104 downward within the patient's mouth and dispose the therapy device underneath the patient's tongue.

In certain embodiments, sublingual light therapy sleeve 104 may be flavored, may contain medications that it self-elutes and may contain one or more photosensitizing agents complimentary to photonic infusion therapy. In certain embodiments, sublingual light therapy sleeve 104 may be made to dissolve during the therapy session. It could be made of silicone or other elastomers, food-grade starch, sugar protein, lipid, or some combination thereof.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refers to at least one of something selected from the group consisting of A, B, C . . . and N, the text should be interpreted as requiring only one element from the group, not A plus N, or B plus N, etc.

1. A sublingual light therapy device comprising:
a light source;
a light conduit having a first end and a second end, connected to the light source at the first end;
a mouthpiece, comprising a sublingual photonic applicator disposed inside a compliant sublingual light therapy sleeve, connected to the second end of the light conduit.

2. The sublingual light therapy device of claim 1, wherein the sublingual photonic applicator comprises a main body having an optical connector on one side thereof.

3. The sublingual light therapy device of claim 2, wherein the sublingual photonic applicator comprises a pair of optical horns opposite the optical connector.

4. The sublingual light therapy device of claim 3, wherein the optical horns are connected to the main body via an optical manifold having a branched “V” shape.

5. The sublingual light therapy device of claim 3, wherein each optical horn has a curved shape and a generally-circular cross-section.

6. The sublingual light therapy device of claim 3, wherein a central aperture runs axially down the center of each optical horn.

7. The sublingual light therapy device of claim 6, wherein each central aperture is sized and shaped to accommodate an optical fiber.

8. The sublingual light therapy device of claim 7, wherein the optical fiber is an 800 μm core fiber optic.

9. The sublingual light therapy device of claim 2, wherein the main body incorporates a generally-cylindrical central core having a generally-rectangular upper landing on the top thereof.

10. The sublingual light therapy device of claim 2, wherein the main body incorporates a pair of prismatic wings extending downwardly from each side thereof.
